(19) United States
(12) Patent Application Publication
(10) Pub. No.: US 2014/0100787 A1
(43) Pub. Date: Apr. 10, 2014

(54) Assay Analysis, Tracking and Reporting System (AATR)

(71) Applicants: AAKASH JHA, OAK PARK, CA (US); AADARSH JHA, OAK PARK, CA (US)

(72) Inventors: AAKASH JHA, OAK PARK, CA (US); AADARSH JHA, OAK PARK, CA (US)

(21) Appl. No.: 13/633,863

(22) Filed: Oct. 9, 2012

(51) Int. Cl. G01N 33/48; G06F 19/00

(52) U.S. Cl. 702/19

(57) ABSTRACT

The Assay Analysis, Tracking and Reporting (AATR) is a software system to acquire and analyse Enzyme-Linked ImmunoSorbant Assays (ELISA) assays for the purspose of Antibody Screening, Detection, Concentration, Tracking, Dose Respone, Adsorption Analysis and Reporting using second/third/fourth degree polynomial, 4 and 5 Parameter Logistics best curve fitting regression analysis. AATR provides mechanism and capabilities to acquire data directly from Microplate Reader devices including batch mode and allows for import/export of ELISA data and sample ids for the tray. The regression analysis uses positive controls and negative controls and multiple standards data points. An average is calcuated for the duplicate samples. The software automatcialy color codes the wells and sample ids in the plate for the outliers data. Additional color coding and marker are used to designate the wells/samples above and below the highest and lowest thresold. AATR allows exporting of graphs, data points and reports in various formats.

### CROSS REFERENCE TO RELATED APPLICATIONS (IF ANY)—

Not Applicable

### STATEMENT OF FEDERALLY SPONSORED RESEARCH/DEVELOPMENT—

Not Applicable

### BACKGROUND OF THE INVENTION—

ELISA assays are used in the clinics, diagnostic labs, medical centers, research centers to analyze the samples and related data for many pursposes. These tests and related analysis and reporting are done differently using different tools in the institutions mentioned above. The use of multipe incosistent tools and method do not produce consistent and very reliable results and interpreation of such results.

The Assay Analysis, Tracking and Reporting (AATR) system is a computer software system to acquire and analyse Enzyme-Linked ImmunoSorbant Assays (ELISA) assays for the purspose of Antibody Screening, Detection, Concentration, Tracking, Dose Respone, Adsorption Analysis and Reporting purposes. The AATR is a database based software which persists the above information including configurations, batch information, sample information, device information, analysis results, thresold etc. to make acciesible to users in standalone as well as network environment.

### BRIEF SUMMARY OF THE INVENTION—

The Assay Analysis, Tracking and Reporting (AATR) system is a computer software system to acquire and analyse Enzyme-Linked ImmunoSorbant Assays (ELISA) assays for the purspose of Antibody Screening, Detection, Concentration, Tracking, Dose Respone, Adsorption Analysis and Reporting purposes. It is a very unique and one of a kind of software system. The AATR is a database based software which persists the above information including configurations, batch information, sample information, device information, analysis results, thresold etc. to make acciesible to users in standalone as well as network environment.

### BRIEF DESCRIPTION OF THE SEVERAL VIEWS OF THE DRAWING (IF ANY)—

a. Description of fields and function

b. Drawing 1—Shows the design and architecture of the software.

c. Drawing 2—Shows the sample, OD values, plate configuration, and Microplate Reader device details.

d. Drawing 3—Shows the regression analysis results and best fit curve

### DETAILED DESCRIPTION OF THE INVENTION

The Assay Analysis, Tracking and Reporting (AATR) system is a computer software system to acquire and analyse Enzyme-Linked ImmunoSorbant Assays (ELISA) assays for the purspose of Antibody Screening, Detection, Concentration, Tracking, Dose Respone, Adsorption Analysis and Reporting purposes using linear and non-linear (e.g. second degree, third degree polynomial, 4PL—4 Parameter Logistics, 5PL—5 Parameter Logistics) best curve fitting regression analysis. The AATR provides mechanism and capabilities to acquire data from Microplate Reader devices in real time as well in batch mode as well as allows for import and export of ELISA data and sample ids for the tray being read. The linear and non-linear regression uses positive controls and negative controls as well as multiple standards data points. The samples are organized in a tray or plate. A plate can have one or more samples. Duplicate samples are allowed though not rquired for better analysis and results. Controls (PC and NC) and 5 Standards values are required. The duplicate controls and standards are also allowed. An average is calcuated for the duplicate samples. The software automatcialy color codes the wells and sample ids in the plate for the out of range data. The additional color coding and marker are used to designate the wells/samples above and below the highest and lowest threspold. The AATR allows exporting of graphs, data points and reports in various format. The AATR is a database based software which persists the above information including configurations, batch information, sample information, device information, analysis results, thresold etc. to make acciesible to users in standalone as well as network environment.

Description of the Fields, Functions on the Screen and in the System

1. Tray ID—The “Tray ID:” allows to create/give a tray name. It is user editable field and value.

2. Plate ID—The “Plate ID:” allows to create/give a plate name. It is user editable field and value.

3. Date—The “Date:” allows to enter the date tray was acquired using “Read ELISA” function described below. By default it take today's date. It is user editable field and value.

4. Filter Used—The “Filter Used:” . . . . It is user editable field and value.

5. Secondary Ab—The “Secondary Ab:” allows to mark the secondary antibody used in the well with the ELISA sample.

6. Negative and Positive Control—The system allows to establish the controls for the ELISA test—
a. The “NC”—Negative Control. Allows for any well to be marked at “NC”.
b. The “PC”—Positive Control. Allows for any well to be marked at “PC”.
c. Other Controls—Other control can be established as well.

7. Standard Units—Allows to establish standard units. These units are configurable as can be changed on test/experiment basis.

8. Standards—Allows to establish standards for the ELISA samples. There are 5 required Standards values and more can be added on test/experiment basis. These standards are configurable using the well position in the tray.

9. Micro plate Reader—Diplays the name of the device.

10. Software Version—Diplays the software version installed on Microplate Redaer device.

11. Plate Type—Diplays the plate name and the geomtry of the plate.

12. Functions and Tabs—There are two tabs—
a. Read File—This display the two plate design of 96 wells each. The first tray shows/display the optical value (observed value) for each sample in a given well. The second plate design shows the sample id by well and each sample corresponde with the well in the first plate. The sample id value can be imported from file using the button “Import Sample ID” or it can be typed in.
b. Graph—This tab and function displays a graph and a grid. Some of the columns such as Well Pos, Sample ID, and O.D. value in the grid is based on the data in the “Read Batch” tab. The rest of the data elements such as Average O.D., Average U/ml, U/ml, and High are calculated using a regression analysis.

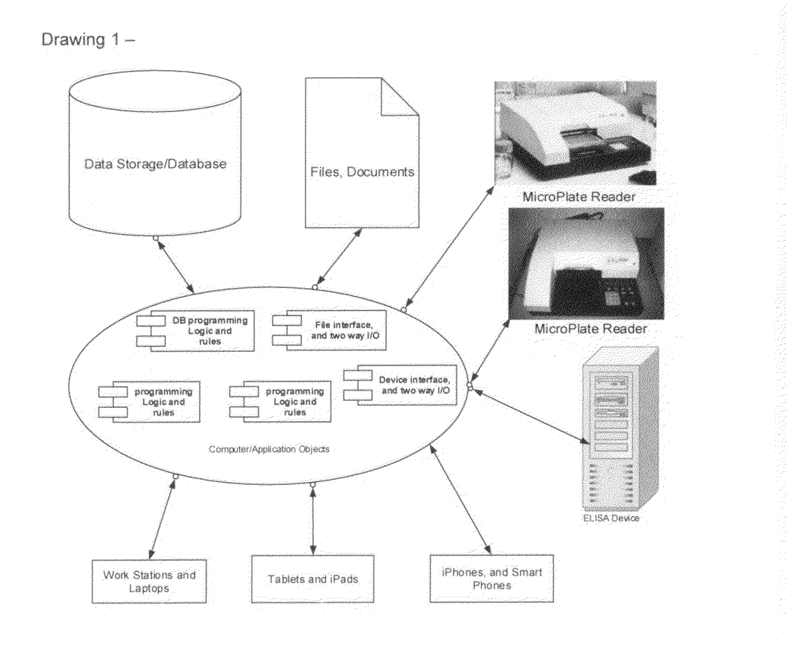

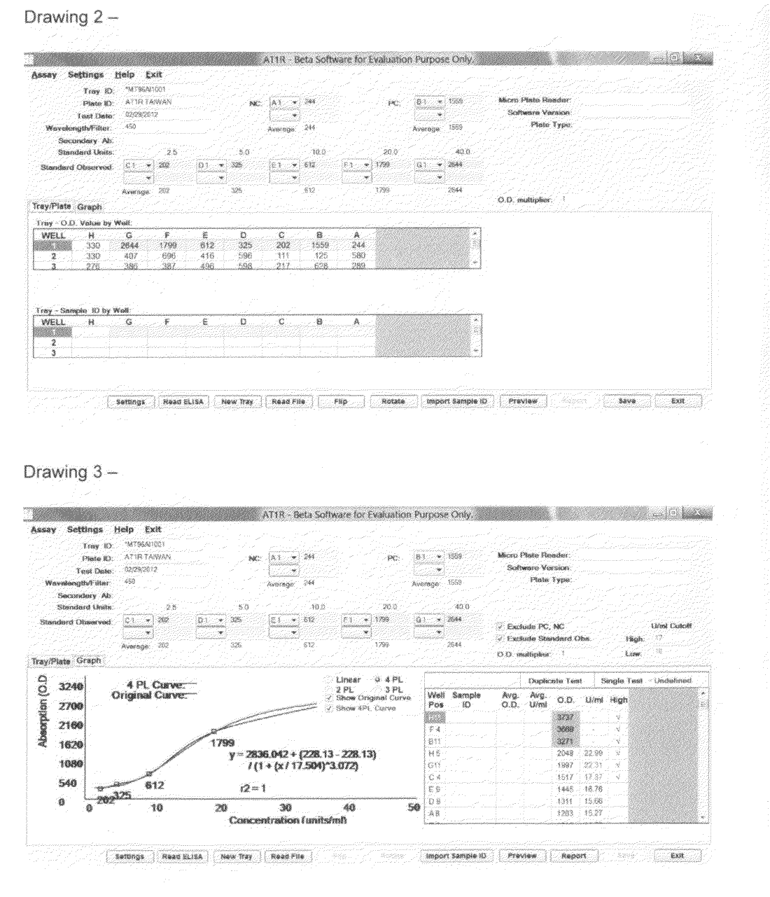

Save—The “Save” function allows to save the plate/tray in a CSV file which can be read later using this foftware. Gives user a choice where to save file as shown in FIG. 4.

Claims—

We claim that the “The Assay Analysis, Tracking and Reporting (AATR)” software system is a unique, innovative and one of a kind computer software system to acquire and analyse Enzyme-Linked ImmunoSorbant Assays (ELISA) assays for the purspose of Antibody Screening, Detection, Concentration, Tracking, Dose Respone, Adsorption Analysis and Reporting purposes using linear and non-linear (e.g. second degree, third degree polynomial, 4PL—4 Parameter Logistics, 5PL—5 Parameter Logistics) best curve fitting regression analysis. The AATR provides mechanism and capabilities to acquire data from Microplate Reader devices in real time as well in batch mode as well as allows for import and export of ELISA data and sample ids for the tray being read. The linear and non-linear regression uses positive controls and negative controls as well as multiple standards data points. An average is calcuated for the duplicate samples. The software automatcialy color codes the wells and sample ids in the plate for the out of range data. The additional color coding and marker are used to designate the wells/samples above and below the highest and lowest threspold. The AATR allows exporting of graphs, data points and reports in various format. The AATR is a database based software which persists the above information including configurations, batch information, sample information, device information, analysis results, thresold etc. to make acciesible to users in standalone as well as network environment.

1. We claim that the “The Assay Analysis, Tracking and Reporting (AATR)” software system is a unique, innovative and one of a kind computer software system to acquire and analyse Enzyme-Linked ImmunoSorbant Assays (ELISA) assays for the purspose of Antibody Screening, Detection, Concentration, Tracking, Dose Respone, Adsorption Analysis and Reporting purposes using linear and non-linear (e.g. second degree, third degree polynomial, 4PL—4 Parameter Logistics, 5PL—5 Parameter Logistics) best curve fitting regression analysis. The AATR provides mechanism and capabilities to acquire data from Microplate Reader devices in real time as well in batch mode as well as allows for import and export of ELISA data and sample ids for the tray being read. The linear and non-linear regression uses positive controls and negative controls as well as multiple standards data points. An average is calcuated for the duplicate samples. The software automatcialy color codes the wells and sample ids in the plate for the out of range data. The additional color coding and marker are used to designate the wells/samples above and below the highest and lowest threspold. The AATR allows exporting of graphs, data points and reports in various format. The AATR is a database based software which persists the above information including configurations, batch information, sample information, device information, analysis results, thresold etc. to make acciesible to users in standalone as well as network environment.
